SEQUENCE LISTING (1 sequence)

SEQ ID NO: 1 - DNA, 20 bp, synthetic construct
  misc_feature 1..20: note = Oligonucleotide
  source 1..20: mol_type = other DNA
ctcagtaacattgacaccac

(19) United States
(12) Patent Application Publication
(10) Pub. No.: US 2023/0322843 A1
(43) Pub. Date: Oct. 12, 2023

(54) Process for the preparation of oligonucleotides using modified oxidation protocol

(71) Applicant: Hoffmann-La Roche Inc., Little Falls, NJ (US)

(72) Inventors: Alec FETTES, Hagendorn (CH); Achim GEISER, Seon (CH); Leonhard JAITZ, Oeschgen (CH)

(73) Assignee: Hoffmann-La Roche Inc., Little Falls, NJ (US)

(21) Appl. No.: 18/150,553

(22) Filed: Jan. 5, 2023

Related U.S. Application Data
(63) Continuation of application No. PCT/EP2021/068832, filed on Jul. 7, 2021.

(30) Foreign Application Priority Data
Jul. 9, 2020 (EP) 20184839.7

(51) Int. Cl. C07H 21/02

(52) U.S. Cl. CPC C07H 21/02

(57) ABSTRACT

The invention relates to a process for the production of a mixed P═O/P═S backbone oligonucleotide comprising a selective oxidation of an intermediary phosphite triester compound of formula I into a phosphodiester compound of formula II according to the scheme

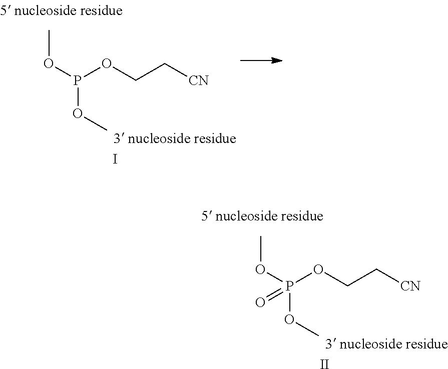

applying a novel oxidation protocol and to new oxidation solutions.

### CROSS REFERENCE TO RELATED APPLICATIONS

This application is a Continuation of International Application No. PCT/EP2021/068832, filed Jul. 7, 2021, which claims the benefit of European Patent Application No. 20184839.7, filed Jul. 9, 2020, each of which is incorporated by reference herein in its entirety.

### REFERENCE TO SEQUENCE LISTING SUBMITTED ELECTRONICALLY

This application incorporates by reference a Sequence Listing electronically submitted in a XML file entitled “P36240-US.xml”, created on Dec. 19, 2022 and having a size of 2,312 bytes.

The invention relates to a novel process for the production of a mixed P═O/P═S backbone oligonucleotide comprising the oxidation of an intermediary phosphite triester compound of formula I into a phosphodiester compound of formula II according to the scheme

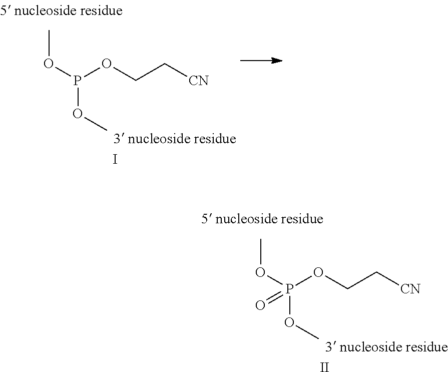

wherein the oxidation makes use of a particular oxidation solution and of novel oxidation solutions.

The oligonucleotide synthesis in principle is a stepwise addition of nucleotide residues to the 5′-terminus of the growing chain until the desired sequence is assembled.

As a rule, each addition is referred to as a synthetic cycle and in principle consists of the chemical reactions

a₁) de-blocking the protected hydroxyl group on the solid support,

a₂) coupling the first nucleoside as activated phosphoramidite with the free hydroxyl group on the solid support,

a₃) oxidizing or sulfurizing the respective P-linked nucleoside (phosphite triester) to form the respective phosphodiester (P═O) or the respective phosphorothioate (P═S);

a₄) optionally, capping any unreacted hydroxyl groups on the solid support; a₅) de-blocking the 5′ hydroxyl group of the first nucleoside attached to the solid support;

a₆) coupling the second nucleoside as activated phosphoramidite to form the respective P-linked dimer;

a₇) oxidizing or sulfurizing the respective P-linked dinucleotide (phosphite triester) to form the respective phosphodiester (P═O) or the respective phosphorothioate (P═S);

a₈) optionally, capping any unreacted 5′ hydroxyl groups;

a₉) repeating the previous steps as to as until the desired sequence is assembled.

The oxidizing step is typically performed with an oxidation solution comprising iodine, an organic solvent, which as a rule is pyridine and water.

However, it was observed that when a freshly prepared oxidation solution has been applied, not only the desired oxidation of the intermediary phosphite triester compound of formula I into a phosphodiester compound of formula II takes place, but also, as a side reaction, phosphorothioate internucleotide linkages present in the molecule may be affected by a P═S to P═O conversion at the internucleotide linkages which resulted in a higher than expected content of phosphodiester linkages within the compound of formula II.

Object of the invention therefore was to find an oxidation protocol which allows a selective oxidation of the phosphite triester compound of formula I into the phosphodiester compound of formula II without affecting the phosphorothioate internucleotide linkage. A further object of the invention was to find an oxidation solution, which can be readily applied when prepared without the need of further treatments such as aging.

It was found that the object of the invention could be reached with the process for the production of a mixed P═O/P═S backbone oligonucleotide which comprises the oxidation of an intermediary phosphite triester compound of formula I into a phosphodiester compound of formula II according to the scheme

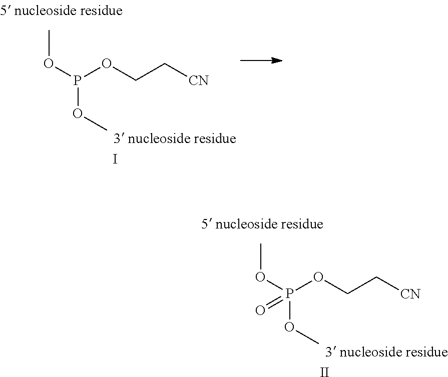

with an oxidation solution containing iodine, an organic solvent and water and which is characterized in that it in addition contains an iodide.

The following definitions are set forth to illustrate and define the meaning and scope of the various terms used to describe the invention herein.

The term “C₁₋₆-alkyl” denotes a monovalent linear or branched saturated hydrocarbon group of 1 to 6 carbon atoms, and in a more particular embodiment 1 to 4 carbon atoms. Typical examples include methyl, ethyl, propyl, isopropyl, n-butyl, i-butyl, sec-butyl, or t-butyl, preferably methyl or ethyl.

The term oligonucleotide as used herein is defined as it is generally understood by the skilled person as a molecule comprising two or more covalently linked nucleotides.

For use as a therapeutically valuable oligonucleotide, oligonucleotides are typically synthesized as 10 to 40 nucleotides, preferably 10 to 25 nucleotides in length.

The oligonucleotides may consist of optionally modified DNA or RNA nucleoside monomers or combinations thereof.

Optionally modified as used herein refers to nucleosides modified as compared to the equivalent DNA or RNA nucleoside by the introduction of one or more modifications of the sugar moiety or the nucleobase moiety.

Typical modifications can be the 2′-O-(2-Methoxyethyl)-substitution (2′-MOE) substitution in the sugar moiety or the locked nucleic acid (LNA), which is a modified RNA nucleotide in which the ribose moiety is modified with an extra bridge connecting the 2′ oxygen and the 4′ carbon.

The term modified nucleoside may also be used herein interchangeably with the term “nucleoside analogue” or modified “units” or modified “monomers”.

The DNA or RNA nucleotides are as a rule linked by a phosphodiester (P═O) or a phosphorothioate (P═S) internucleotide linkage which covalently couples two nucleotides together.

In accordance with the invention at least one internucleotide linkage has to consist of a phosphorothioate (P═S). Accordingly, in some oligonucleotides all other internucleotide linkages may consist of a phosphodiester (P═O) or in other oligonucleotides the sequence of internucleotide linkages vary and comprise both phosphodiester (P═O) and phosphorothioate (P═S) internucleotide linkages.

Accordingly the term mixed P═O/P═S backbone oligonucleotide refers to oligonucleotides wherein at least one internucleotide linkage has to consist of a phosphorothioate (P═S) and at least one internucleotide linkage consists of a phosphodiester (P═O).

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are described with capital letters A, T, G and ^{Me}C (5-methyl cytosine) for LNA nucleoside and with small letters a, t, g, c and ^{Me}C for DNA nucleosides. Modified nucleobases include but are not limited to nucleobases carrying protecting groups such as tert-butylphenoxyacetyl, phenoxyacetyl, benzoyl, acetyl, isobutyryl or dimethylformamidino (see Wikipedia, Phosphoramidit-Synthese, https://de.wikipedia.org/wiki/Phosphoramidit-Synthese of Mar. 24, 2016).

Preferably the oligonucleotide consists of optionally modified DNA or RNA nucleoside monomers or combinations thereof and is 10 to 40, preferably 10 to 25 nucleotides in length.

The principles of the oligonucleotide synthesis are well known in the art (see e.g.

Oligonucleotide synthesis; Wikipedia, the free encyclopedia; https://en.wikipedia.org/wiki/Oligonucleotide synthesis, of Mar. 15, 2016).

Larger scale oligonucleotide synthesis nowadays is carried out in an automated manner using computer-controlled synthesizers.

As a rule, oligonucleotide synthesis is a solid-phase synthesis, wherein the oligonucleotide being assembled is covalently bound, via its 3-terminal hydroxy group, to a solid support material and remains attached to it over the entire course of the chain assembly. Suitable supports are the commercial available macroporous polystyrene supports like the Primer support 5G from GE Healthcare or the NittoPhase®HL support from Kinovate.

The subsequent cleavage from the resin can be performed with concentrated aqueous ammonia. The protecting groups on the phosphate and the nucleotide base are also removed within this cleavage procedure.

As outlined above the process for the production of a mixed P═O/P═S backbone oligonucleotide is comprising the oxidation of an intermediary phosphite triester compound of formula I into a phosphodiester compound of formula II.

The oxidation solution can be prepared by mixing the iodide with water and the organic solvent and by the subsequent addition of iodine.

The iodide can be selected from hydrogen iodide, from an alkali-iodide or from an alkali-tri-iodide, preferably from hydrogen iodide or from an alkali-iodide, more preferably from sodium- or potassium iodide.

The organic solvent can be selected from pyridine or from a C₁₋₆ alkyl-substituted pyridine e.g. lutidine, but preferably from pyridine. A further organic solvent such as tetrahydrofuran may be present.

The volume ratio organic solvent to water is as a rule selected from 1:1 to 20:1, preferably from to 5:1 to 15:1, more preferably is 9:1.

The molar ratio of iodine to iodide in the oxidation solution is selected in the range of 1.0:0.1 to 1.0:3.0, preferably 1.0:1.0 to 1.0:2.0.

The iodine concentration in the oxidation solution is typically applied in the range of 10 mM to 100 mM, preferably of 15 mM to 60 mM.

Based on an iodine content of 50 mM, iodide is added in an amount until the oxidation solution has a conductivity of ≥1500 S/cm.

In a preferred embodiment the iodide is potassium iodide and the oxidation solution has a conductivity, on the basis of a content of 50 mM KI and 50 mM I₂, of ≥1500 S/cm, preferably between 1650 and 2050 S/cm., more preferably between 1750 and 1950 S/cm.

Based on an iodine content of 10 mM, iodide is added in an amount until the oxidation solution has a conductivity of ≥300 μS/cm.

In a preferred embodiment, the iodide is potassium iodide and the oxidation solution has a conductivity on the basis of 10 mM KI and 10 mM 12; of ≥300 S/cm, preferably between 350 and 550 S/cm, more preferably between 400 and 500 S/cm.

Based on a iodine content of 20 mM, iodide is added in an amount until the oxidation solution has a conductivity of ≥600 S/cm.

In a preferred embodiment, the iodide is potassium iodide and the oxidation solution has a conductivity on the basis of 20 mM KI and 20 mM 12; of ≥600 S/cm, preferably between 750 and 950 S/cm., more preferably between 800 and 900 S/cm.

Based on an iodine content of 100 mM, iodide is added in an amount until the oxidation solution has a conductivity of ≥3000 S/cm.

In a preferred embodiment, the iodide is potassium iodide and the oxidation solution has a conductivity on the basis of 100 mM KI and 100 mM I₂; of ≥3000 S/cm, preferably between 3200 and 3900 S/cm, more preferably between 3350 and 3750 S/cm.

Typically the oxidation solution is capable to oxidize the intermediary phosphite triester compound of formula I into the phosphodiester compound of formula II in such a manner that the P═O content in the reaction solution reaches a value below 2.5%, preferably below 2.0%.

Aa a further embodiment of the present invention a method for assessing the quality of an oxidation solution is provided which comprises
a) providing an oxidation solution comprising iodine an organic solvent and water,
b) measuring the conductivity of the oxidation solution and
c) based on a certain threshold value of the measured conductivity assessing the suitability of the oxidation solution for oxidizing the intermediary phosphite triester compound of formula I into the phosphodiester compound of formula II.

As a further, more preferred embodiment of the method for assessing the quality of an oxidation solution, the oxidation solution in addition comprises an iodide.

The amount of iodine used for the preparation of the oxidation reaction is usually selected between 1.1 equivalents and 15 equivalents, more preferably between 1.5 equivalents and 4.5 equivalents.

The oxidation reaction is performed between 15° C. and 27° C., more preferably between 18° C. and 24° C.

As outlined above, with the preferred embodiment of the invention, i.e. with stoichiometric ratios of iodine and iodide or ratios where an excess iodide is present the oxidation solution can immediately be applied after its preparation.

In another, however less preferred, embodiment of the invention ratios of iodine and iodide with substoichiometric amounts of iodide can be used.

Such oxidation solutions may require a certain time of aging until they have the required properties, in terms of conductivity and of the potential to selectively oxidize the phosphite triester compound of formula I into the phosphodiester compound of formula II.

The optimal period for the aging is largely determined by the temperature at which the oxidation solution is aged. While a low aging temperature results in a longer aging period, a higher aging temperature significantly reduces the aging time.

For instance, the oxidation solution can be aged at a temperature of 20° C. to 100° C., but preferably at a temperature of 30° C. to 60° C.

The time period required for the aging of the oxidation solution has to be sufficient to effect selective oxidation of the phosphite triester compound of formula I into the phosphodiester compound of formula II without affecting the phosphorothioate internucleotide linkages.

As a rule the oxidation solution can be aged for a time period of at least 1 day, 3 days, 5 days, 10 days, 15 days or at least 20 days.

The time period may, as mentioned, largely vary depending on the aging temperature and for an aging temperature of 30° C. to 35° C. can vary between 10 days and 150 days, more typically between 20 days and 60 days, while for an aging temperature of 60° C. to 65° C. can vary between 1 day and 30 days, more typically between 2 and 15 days.

The aging as a rule goes along with an increase of the conductivity (μS/cm) and a decrease of the pH until a certain plateau is reached.

In a further embodiment the invention comprises new oxidation solutions which may comprise:
a) 10 to 100 mM iodine
b) 0.1 to 3.0 mol eq. of an iodide related to 1.0 mol eq of iodine
c) an organic solvent and
d) water, wherein the volume ratio organic solvent to water; is 20:1 to 1:1 preferably,
a) 15 to 60 mM in iodine
b) 1.0 to 2.0 mol eq. of an iodide related to 1.0 mol eq. of iodine
c) an organic solvent and
d) water, wherein the volume ratio organic solvent to water is 5:1 to 15:1 more preferably,
a) 15 to 60 mM in iodine
b) 1.0 to 2.0 mol eq. of hydrogen iodide or of an alkali iodide related to 1.0 mol eq. of iodine
c) pyridine and
d) water, wherein the volume ratio pyridine to water is 5:1 to 15:1. even more preferably,
a) 15 to 60 mM in iodine
b) 1.0 to 2.0 mol eq. of sodium- or potassium iodide related to 1.0 mol eq of iodine
c) pyridine and
d) water, wherein the volume ratio pyridine to water is 9:1.

By way of illustration the oligonucleotide can be selected from:

5′- ^{Me}C_{S} ^{Me}U_{O} ^{Me}C_{O}A_{O}G_{S}T_{S}A_{S}A_{S} ^{Me}C_{S}A_{S}T_{S}T_{S}G_{S}A_{S} ^{Me}C_{S}A_{O}
^{Me}C_{O} ^{Me}C_{O}A_{S} ^{Me}C- 3′

The underlined residues are 2′-MOE nucleosides. The locations of phosphorothioate and phosphate diester linkages are designated by S and O, respectively. It should be noted that 2′-O-(2-methoxyethyl)-5-methyluridine (2′-MOE MeU) nucleosides are sometimes referred to as 2′-O-(2-methoxyethyl)ribothymidine (2′-MOE T).

The compounds disclosed herein have the following nucleobase sequences

SEQ ID No. 1:
cucagtaacattgacaccac

### EXAMPLES

Synthesis of

5′- ^{Me}C_{S} ^{Me}U_{O} ^{Me}C_{O}A_{O}G_{S}T_{S}A_{S}A_{S} ^{Me}C_{S}A_{S}T_{S}T_{S}G_{S}A_{S} ^{Me}C_{S}A_{O}
^{Me}C_{O} ^{Me}C_{O}A_{S} ^{Me}C-3′

The oligonucleotide was produced by standard phosphoramidite chemistry on solid phase at a scale of 2.20 mmol using an AKTA Oligopilot 100 and Primer Support Unylinker (NittoPhase LH Unylinker 330). In general 1.4 equiv of the DNA/MOE-phosphoramidites were employed. Other reagents (dichloroacetic acid, 1-methylimidazole, 4,5-dicyanoimidazole, acetic anhydride, phenylacetyl disulfide, pyridine, triethylamine) were used as received from commercially available sources and reagent solutions at the appropriate concentration were prepared (see details below). The oxidizer solution was freshly prepared (see below). Cleavage and deprotection was achieved using ammonium hydroxide to give the crude oligonucleotide.

Standard Reagent Solutions

| | |
|---|---|
| Deblock | 10% dichloroacetic acid in toluene (v/v) |
| Phosphoramidites | 0.2M in acetonitrile |
| NMI/DCI activator | 1.0M 4,5-dicyanoimidazole/0.1M |
| | 1-methylimidazole in acetonitrile |
| Thiolation | 0.2M phenylacetyl disulfide in 3- |
| | picoline/acetonitrile (1:1 v/v) |
| Cap A | 1-Methylimidazole/pyridine/acetonitrile |
| | 2:3:5 (v/v/v) |
| Cap B | Acetic anhydride/acetonitrile 1:4 (v/v) |
| Amine wash | 50% triethylamine in acetonitrile (v/v) |
| Cleavage and Deprotection | 28-32% aqueous ammonium hydroxide |

Preparation of Iodine/Potassium Iodide Solution

Potassium iodide was added to water at room temperature, followed by pyridine. Iodine was added and the mixture was stirred for 1 h under a positive pressure of dry nitrogen before being used.

| | 50 mM I₂ | 50 mM I₂ | 50 mM I₂ | 50 mM I₂ | 10 mM I₂ | 20 mM I₂ | 100 mM I₂ |
|---|---|---|---|---|---|---|---|
| | 100 mM KI | 50 mM KI | 25 mM KI | 5 mM KI | 10 mM KI | 20 mM KI | 100 mM KI |
| Amount KI [g] | 16.6 | 8.30 | 4.15 | 0.83 | 0.17 | 0.33 | 16.6 |
| Amount iodine [g] | 12.7 | 12.7 | 12.7 | 12.7 | 0.25 | 0.51 | 25.4 |
| Amount water [g] | 100 | 101 | 101 | 101 | 9.98 | 9.90 | 102 |
| Amount pyridine | 884 | 885 | 887 | 887 | 88.0 | 87.6 | 875 |
| [g] | | | | | | | |

Preparation of Iodine/Sodium Iodide Solution

7.49 g sodium iodide were added to 101 g of water at room temperature, followed by 886 g of pyridine. 12.7 g of iodine were added and the mixture was stirred for 1 h under a positive pressure of dry nitrogen before being used.

Oxidation Examples Using Different Oxidizer Solutions without Aging

| Oxidizer solution | | | Conductivity of |
|---|---|---|---|
| Pyridine: H₂O 9:1 | Total (P═O)₁¹ | pH of oxidizer | oxidizer solution |
| (v/v) | content (%) | solution | [μS/cm] |
| 100 mM I₂ | 1.4 | 9.3 | 3520 |
| 100 mM KI | | | |
| 75 mM I₂ | 1.3 | 9.1 | 2743 |
| 75 mM KI | | | |
| 50 mM I₂ | 1.3 | 9.3 | 3343 |
| 100 mM KI | | | |
| 50 mM I₂ | 1.5 | 8.4 | 1881 |
| 50 mM KI | | | |
| 50 mM I₂ | 10.3 | 8.1 | 1018 |
| 25 mM KI | | | |
| 50 mM I₂ | 15.3 | 7.3 | 382 |
| 5 mM KI | | | |
| 50 mM I₂ | 1.6 | 8.4 | 1806 |
| 50 mM NaI | | | |
| 10 mM I₂ | — | 8.4 | 439 |
| 10 mM KI | | | |
| 20 mM I₂ | — | 8.6 | 830 |
| 20 mM KI | | | |

¹refers to the percentage of molecules having a mass difference of 16 Da relative to the molecular mass of the desired compound determined in mass spectrometry, i.e. percentage of those molecule wherein 1 P═S linkage has been transformed into a P═O linkage.

Aging of KI (50 mM)/I₂ (50 mM) Solution at 30-35° C.

The solution was stored at 30-35° C. in amber glass bottles until use.

Oxidation Examples Using Aged (at 30-35° C.) KI (50 mM)/12 (50 mM) Solutions

| | | | Conductivity of |
|---|---|---|---|
| Age of | Total (P═O)₁¹ | pH of oxidizer | oxidizer solution |
| solution (d) | content (%) | solution | [μS/cm] |
| 0 | 1.7 | 8.9 | 1838 |
| 1 | 1.5 | 8.0 | 1866 |
| 9 | 1.6 | 7.6 | 1920 |
| 17 | 1.6 | 7.6 | 1905 |
| 29 | 1.6 | 7.6 | 1935 |
| 90 | 1.2 | 7.5 | 1979 |
| 176 | 1.4 | 7.4 | 1988 |

¹refers to the percentage of molecules having a mass difference of 16 Da relative to the molecular mass of the desired compound determined in mass spectrometry, i.e. percentage of those molecule wherein 1 P═S linkage has been transformed into a P═O linkage.

1. A process for the production of a mixed P═O/P═S backbone oligonucleotide comprising the oxidation of an intermediary phosphite triester compound of formula I into a phosphodiester compound of formula II: 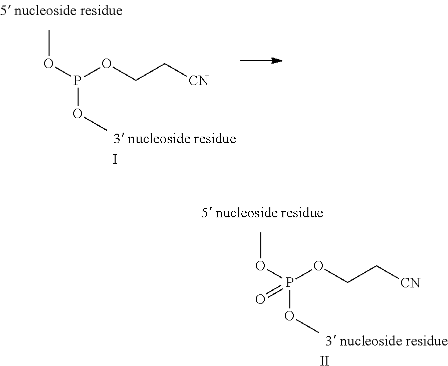
with an oxidation solution comprising iodine, an organic solvent and water, wherein the oxidation solution further comprises iodide.

2. The process of claim 1, wherein the iodide is hydrogen iodide, an alkali-iodide or an alkali-triiodide.

3. (canceled)

4. The process of claim 1, wherein the iodide is an alkali iodide.

5. The process of claim 1, wherein the molar ratio of the iodine to the iodide in the oxidation solution is between 1.0:0.1 and 1.0:3.0.

6. The process of claim 1, wherein the organic solvent is pyridine or a C₁₋₆ alkyl-substituted pyridine.

7. The process of claim 6, wherein the organic solvent is pyridine.

8. The process of claim 1, wherein the volume ratio of the organic solvent to water is from 1:1 to 20:1.

9. The process of claim 1, wherein the concentration of the iodine in the oxidation solution is 10 mM to 100 mM.

10. (canceled)

11. The process of claim 1, wherein the oxidation solution comprises:
(a) 50 mM KI and 50 mM I₂, and the oxidation solution has a conductivity of between 1650 and 2050 μS/cm;
(b) 10 mM KI and 10 mM I₂, and the oxidation solution has a conductivity of between 350 and 550 μS/cm;
(c) 20 mM KI and 20 mM I₂, and the oxidation solution has a conductivity of between 750 and 950 μS/cm; or
(d) 100 mM KI and 100 mM I₂, and the oxidation solution has a conductivity of between 3200 and 3900 μS/cm.

12-17. (canceled)

18. The process of claim 1, wherein the oxidation solution is capable to oxidize the intermediary phosphite triester compound of formula I into the phosphodiester compound of formula II in such a manner that the P═O content in the reaction solution reaches a value below 2.5%.

19. The process of claim 1, wherein the amount of iodine used for the preparation of the oxidation solution is selected between 1.1 equivalents and 15 equivalents to the iodine.

20. The process of claim 1, wherein the reaction temperature for the oxidation reaction is between 15° C. and 27° C.

21. The process of claim 1, wherein the oligonucleotide is an optionally modified DNA or RNA nucleoside monomers or combinations thereof and is 10 to 40, preferably 10 to 25 nucleotides in length.

22. A solution for oxidation, comprising:
a) 10 to 100 mM iodine
b) 0.1 to 3.0 mol eq. of an iodide related to 1.0 mol eq of iodine
c) an organic solvent and
d) water, wherein the volume ratio organic solvent to water; is 20:1 to 1:1.

23. (canceled)

24. The solution of claim 22, comprising:
a) 15 to 60 mM iodine
b) 1.0 to 2.0 mol eq. of hydrogen iodide or of an alkali iodide related to 1.0 mol eq. of iodine
c) pyridine and
d) water, wherein the volume ratio organic solvent to water is 5:1 to 15:1.

25. A method for assessing the quality of an oxidation solution, comprises
a) providing an oxidation solution comprising iodine, an organic solvent, and water;
b) measuring the conductivity of the oxidation solution and
c) based on a certain threshold value of the measured conductivity aassessing the suitability of the oxidation solution for oxidizing the intermediary phosphite triester compound of formula I into the phosphodiester compound of formula II:

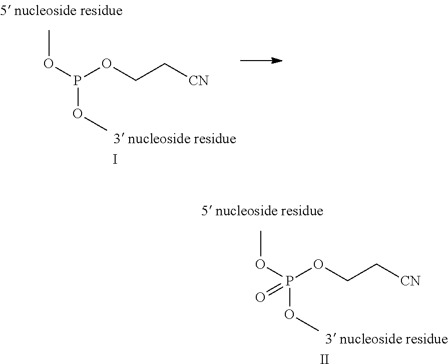

26. (canceled)

27. The process of claim 5, wherein the molar ratio of the iodine to the iodide in the oxidation solution between 1.0:1.0 and 1.0:2.0.

28. The process of claim 8, wherein the volume ratio of the organic solvent to water is about 9:1.

29. The process of claim 9, wherein the concentration of the iodine in the oxidation solution is 15 mM to 60 mM.

30. The process of claim 18, wherein the oxidation solution is capable to oxidize the intermediary phosphite triester compound of formula I into the phosphodiester compound of formula II in such a manner that the P═O content in the reaction solution is below 2.0%.
